(19) United States
(12) Patent Application Publication
(10) Pub. No.: US 2025/0359738 A1
(43) Pub. Date: Nov. 27, 2025

(54) ENDOSCOPE

(71) Applicant: FUJIFILM Corporation, Tokyo (JP)

(72) Inventor: Yuto SHIMOYAMA, Ashigarakami-gun (JP)

(73) Assignee: FUJIFILM Corporation, Tokyo (JP)

(21) Appl. No.: 19/214,087

(22) Filed: May 21, 2025

(30) Foreign Application Priority Data
May 24, 2024 (JP) 2024-085059

(51) Int. Cl. A61B 1/005; A61B 1/018

(52) U.S. Cl. CPC A61B 1/0055; A61B 1/018; A61B 2560/0462

(57) ABSTRACT

An endoscope includes: an insertion part including a distal end part that includes an ultrasound transducer array, and a bendable part that is provided on a proximal end side of the distal end part; and a bending operation part that is capable of performing a bending operation on the bendable part, the bendable part includes a first region, and a second region between the first region and the distal end part, the first region is bent toward a side of a surface of the distal end part on which the ultrasound transducer array is provided in a case where the bending operation part is operated in a first range, and the second region is held in a state of extending along an axis of the distal end part in a case where the bending operation part is operated in the first range.

### CROSS-REFERENCE TO RELATED APPLICATION

The present application claims priority under 35 U.S.C. § 119 to Japanese Patent Application No. 2024-085059 filed on May 24, 2024. The above application is hereby expressly incorporated by reference, in its entirety, into the present application.

### BACKGROUND OF THE INVENTION

### 1. Field of the Invention

The present invention relates to an endoscope.

### 2. Description of the Related Art

JP2005-334050A, JP1990-271817A (JP-H02-271817A), JP1981-124401A (JP-S56-124401A), and JP1999-155806A (JP-H11-155806A) disclose an endoscope having a bendable part that allows a distal end portion to be bent more than a proximal end portion.

JP2004-154545A describes an endoscope having a bendable part which is bendable with a plurality of curvature radii.

JP2000-342517A describes that in a bendable part structure of an endoscope configured by connecting a plurality of nodal rings along an axial direction of an insertion part, the plurality of nodal rings are connected such that an interval between adjacent nodal ring bodies gradually increases from a proximal end part side of the bendable part toward a distal end part side of the bendable part.

### SUMMARY OF THE INVENTION

In the technology of the present disclosure, an endoscope capable of appropriately bringing a part of an ultrasound transducer array into contact with a target part is provided.

An endoscope according to an aspect of the present disclosed technology comprises an insertion part including a distal end part that includes an ultrasound transducer array, and a bendable part that is provided on a proximal end side of the distal end part; and a bending operation part that is capable of performing a bending operation on the bendable part, in which the bendable part includes a first region, and a second region between the first region and the distal end part, the first region is bent toward a surface side of the distal end part on which the ultrasound transducer array is provided in a case where the bending operation part is operated in a first range, and the second region is held in a state of extending along an axis of the distal end part in a case where the bending operation part is operated in the first range.

According to the technology of the present disclosure, it is possible to appropriately bring a part of the ultrasound transducer array into contact with the target part.

### BRIEF DESCRIPTION OF THE DRAWINGS

FIG. 1 is an overall view of an ultrasound endoscope 1 as an endoscope of an aspect of the technology of the present disclosure.

FIG. 2 is a perspective view of a distal end hard part 34 of a first embodiment.

FIG. 3 is an exploded perspective view of the distal end hard part 34 of the first embodiment.

FIG. 4 is a cross-sectional view of the distal end hard part 34 of the first embodiment.

FIG. 5 is a perspective view of the distal end hard part 34 including a tangent line LT.

FIG. 6 is a side view of the distal end hard part 34 including the tangent line LT.

FIG. 7 is a cross-sectional view of a distal end hard part 34 of a modification example.

FIG. 8 is a perspective view schematically showing a main part of a bendable part 32 of the endoscope 1 shown in FIG. 1.

FIG. 9 is an enlarged perspective view of a first movable member 32A shown in FIG. 8.

FIG. 10 is an enlarged perspective view of a second movable member 32B shown in FIG. 8.

FIG. 11 is a view schematically showing a state in which the bendable part 32 shown in FIG. 8 is viewed in a direction X.

FIG. 12 is a schematic view for illustrating a bent state of the bendable part 32 in a case where an angle lever 16 is rotated in an A1 direction from the state shown in FIG. 11.

FIG. 13 is a schematic view for illustrating a state in which the endoscope 1 is inserted into a bronchus and used.

FIG. 14 is a schematic view for illustrating a bent state of the bendable part 32 in a case where a rotation angle of the angle lever 16 is increased from a state shown in FIG. 12.

FIG. 15 is a schematic view for illustrating a state in which the endoscope 1 is inserted into the bronchus and used.

FIG. 16 is a schematic view for illustrating a bent state of the bendable part 32 in a case where the angle lever 16 is rotated in a −A1 direction from the state shown in FIG. 11.

FIG. 17 is a schematic view for illustrating another state in which the endoscope 1 is used by being inserted into the bronchus.

### DESCRIPTION OF THE PREFERRED EMBODIMENTS

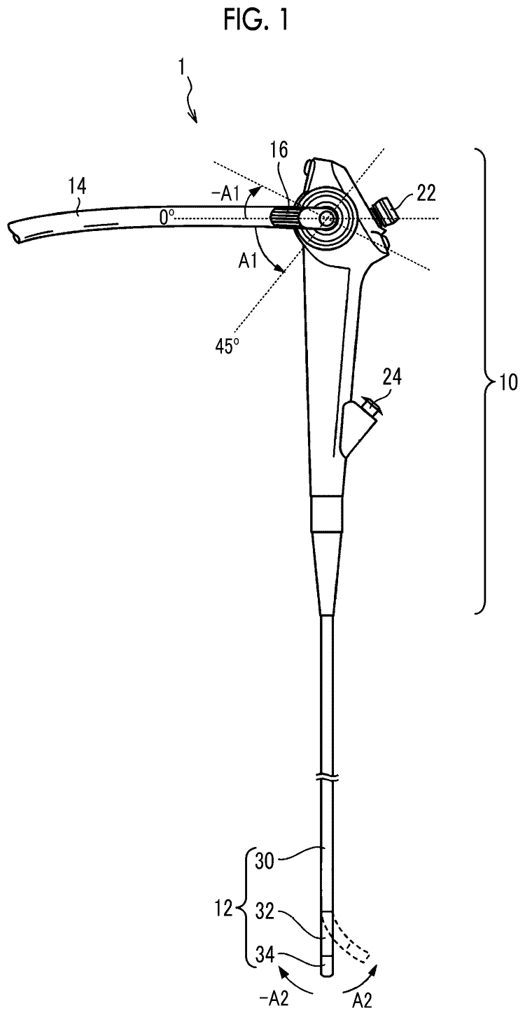

FIG. 1 is an overall view of an ultrasonic endoscope 1 as an endoscope of an aspect of the technology of the present disclosure. As shown in FIG. 1, the ultrasonic endoscope 1 (hereinafter, simply referred to as an “endoscope 1”) is used for collection or the like of a cellular tissue of a lesion part (an observation site, a test site, or an examination site can be used). In the present embodiment, description will be provided in connection with a lymph node of a bronchus as an example of a lesion part.

The endoscope 1 is configured with an operating part 10 that is gripped by a practitioner to perform various operations, an insertion part 12 that is inserted into a body of a patient, and a universal cord 14. The endoscope 1 is connected to system constituent devices that configure an endoscope system, such as a processor device and a light source device (not shown), through the universal cord 14.

The operating part 10 is provided with various operation members that are operated by the practitioner. For example, an angle lever 16, a suction button 22, and the like of which the operations will be appropriately described below are provided.

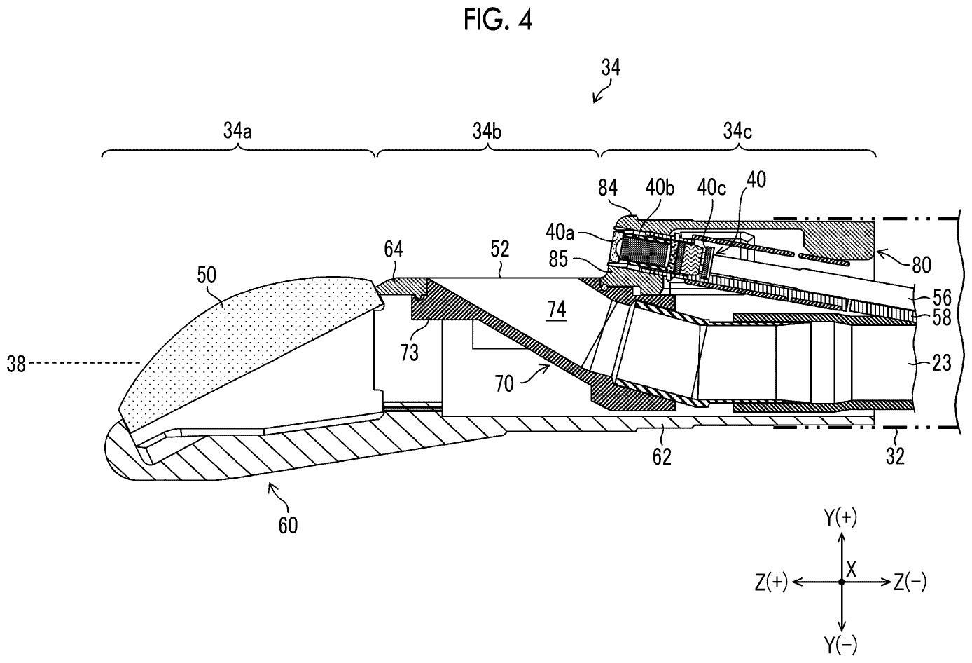

The operating part 10 is provided with a treatment tool inlet port 24 through which a treatment tool is inserted into a treatment tool insertion channel 23 (see FIG. 4) that is inserted into the insertion part 12.

The insertion part 12 extends from a distal end of the operating part 10 and is formed in a small-diameter elongated shape as a whole. The insertion part 12 is configured to be provided with a soft part 30, a bendable part 32, and a distal end hard part 34 which is a distal end part in order from a proximal end side to a distal end side.

The soft part 30 occupies most of the insertion part 12 from the proximal end side and has enough flexibility to be bent in any direction. In a case where the insertion part 12 is inserted into a body cavity, the soft part 30 is bent along an insertion path into the body cavity.

The bendable part 32 is bent in an up-down direction (an A2 direction and a −A2 direction opposite to the A2 direction) by rotationally operating the angle lever 16 of the operating part 10 in the A1 direction and the −A1 direction opposite to the A1 direction, and the distal end hard part 34 can be directed in a desired direction by bending the bendable part 32.

The angle lever 16 is configured to be rotated up to a rotation angle of 45° in the A1 direction with the state shown in FIG. 1 as a rotation angle of 0°, and the bendable part 32 is bent in the A2 direction (upward direction) by rotating the angle lever 16 in the A1 direction. The angle lever 16 is configured to be rotated up to an angle (for example, 25°) less than, for example, a rotation angle of 45° in the −A1 direction, and the bendable part 32 is bent in the −A2 direction (downward direction) by rotating the angle lever 16 in the −A1 direction. The angle lever 16 constitutes a bending operation part that can perform a bending operation of the bendable part 32.

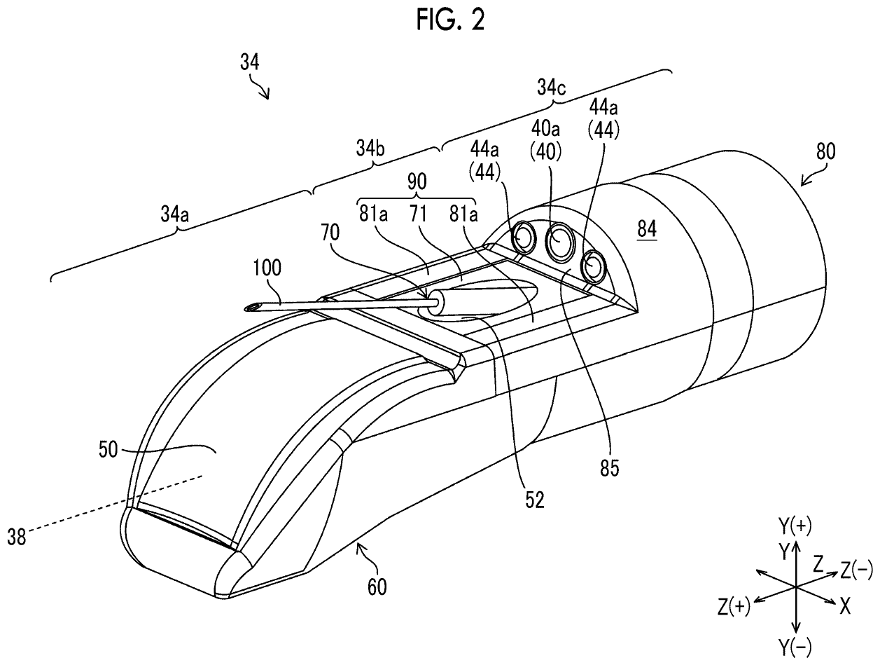

As will be described in detail below referring to FIGS. 2 to 4, the distal end hard part 34 comprises an observation optical system 40 and illumination optical systems 44 that are provided to capture an observation image in the body, an ultrasound transducer 50 that acquires an ultrasound image, and an outlet port 52 from which the treatment tool inserted from the treatment tool inlet port 24 is led out.

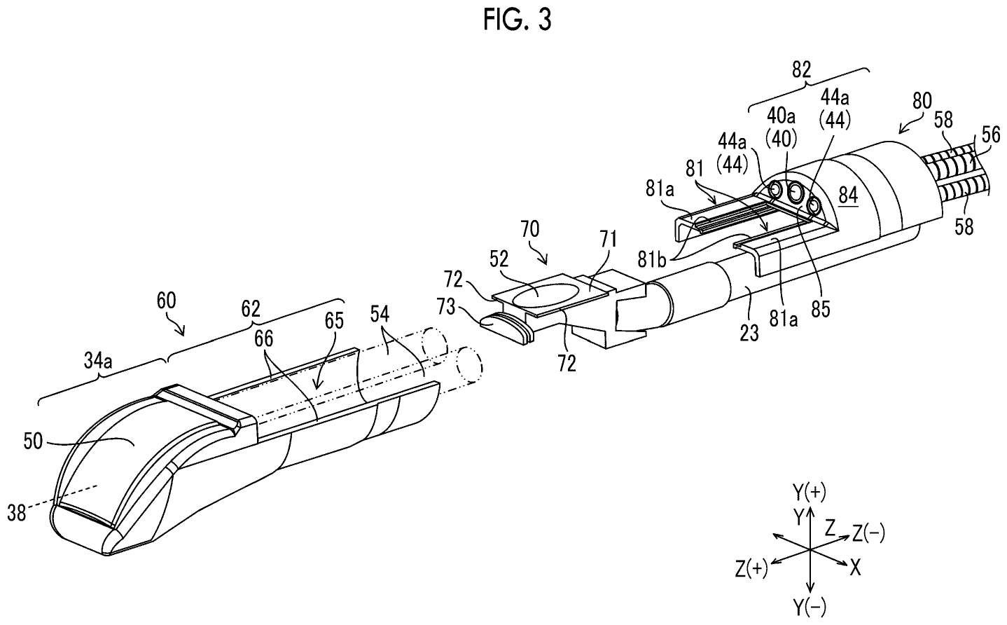

The universal cord 14 includes signal cables 54, a signal cable 56, and light guides 58 shown in FIGS. 3 and 4 described below in detail. A connector is provided in an end part (not shown) of the universal cord 14. The connector is connected to predetermined system constituent devices that configure the endoscope system, such as a processor device and a light source device. As a result, power, control signals, illumination light, and the like necessary for the operation of the endoscope 1 are supplied from the system constituent devices to the endoscope 1. Conversely, data of the observation image acquired by the observation optical system 40 and data of the ultrasound image acquired by the ultrasound transducer 50 are transmitted from the endoscope 1 to the system constituent devices. The observation image and the ultrasound image transmitted to the system constituent devices are displayed on a monitor, and the practitioner or the like can observe the images.

The configuration of the operating part 10 is not limited to the aspect shown in FIG. 1. Instead of the angle lever 16, an angle knob may be provided, or a pair of angle levers 16 or a pair of angle knobs may be provided, and the bending operation of the bendable part 32 may be performed in the up-down direction (A2 direction and −A2 direction) and a left-right direction orthogonal to the up-down direction by performing a rotation operation on the pair of angle levers 16 or the pair of angle knobs. An air/water supply button may be provided in the operating part 10, and gas, such as air, a liquid for cleaning, and the like may be supplied to the distal end hard part 34 by operating the air/water supply button.

FIG. 2 is a perspective view of the distal end hard part 34. FIG. 3 is an exploded perspective view of the distal end hard part 34. FIG. 4 is a cross-sectional view of the distal end hard part 34.

AZ direction in the drawing is a direction parallel to a longitudinal axis 38 of the insertion part 12. The Z direction constitutes a longitudinal axis direction of the insertion part 12. A Z(+) direction side, which is one side of the Z direction in the drawing, is a distal end side of the insertion part 12, and a Z(−) direction side is a proximal end side of the insertion part 12. A Y direction in the drawing is a first direction perpendicular to the Z direction (in other words, perpendicular to the longitudinal axis 38) and corresponds to an A2 direction and a −A2 direction in FIG. 1. A Y(+) direction, which is one side of the Y direction, is the A2 direction in FIG. 1, and a Y(−) direction, which is the other side of the Y direction, is the −A2 direction in FIG. 1. An X direction in the drawing is a second direction perpendicular to both the Z direction and the Y direction.

As shown in FIGS. 2 to 4, the distal end hard part 34 is configured by combining an ultrasound block component 60, a channel block component 70, and an optical system block component 80 (in particular, see FIG. 3). The distal end hard part 34 comprises an ultrasonic attachment part 34a, an outlet port forming part 34b, and a body part 34c from the distal end side toward the proximal end side of the distal end hard part 34 in a state in which the respective block components are combined (see FIGS. 2 and 4).

The ultrasound block component 60 is formed of an insulating material having insulation, and specifically, a resin material, for example, plastic, such as polysulphone and polyether imide. The ultrasound block component 60 comprises the ultrasonic attachment part 34a and an optical system block component attachment part 62 from a distal end side toward a proximal end side thereof (see FIG. 3). The ultrasonic attachment part 34a and the optical system block component attachment part 62 are formed integrally.

The ultrasound transducer 50 is attached to the ultrasonic attachment part 34a in a posture tilted forward (inclined) to the Y(−) direction side with respect to the longitudinal axis 38 in a case where the distal end hard part 34 is viewed from the X direction side. The ultrasound transducer 50 is a convex type that has an ultrasonic wave transmitting and receiving surface on which ultrasound oscillators that transmit and receive ultrasonic waves are arranged in a curved shape along a direction of the longitudinal axis 38. Data for generating an ultrasound image of a lymph node is acquired by the ultrasound transducer 50. The ultrasound transducer 50 constitutes an ultrasound transducer array. The number of ultrasound oscillators that configure the ultrasound transducer 50 is not limited.

The optical system block component attachment part 62 extends from a region on the Y(−) direction side of the proximal end part of the ultrasonic attachment part 34a toward the proximal end side [Z(−) direction side] in a case where the distal end hard part 34 is viewed from the X direction side. A locking part 64 that locks a locked part 73 of the channel block component 70 described below is formed in a region on the Y(+) direction side of the proximal end part of the ultrasonic attachment part 34a (see FIG. 4). The locking part 64 has a locking claw that configures a snap fit, for example.

The optical system block component attachment part 62 has a substantially semi-cylindrical shape corresponding to a divided part on the Y(−) direction side, that is, a divided part on a lower half side out of two divided parts obtained by dividing the outlet port forming part 34b and the body part 34c into two parts in the Y direction (into two parts vertically) (see FIG. 3). For this reason, the optical system block component attachment part 62 has an attachment part opening 65 that is opened on the Y(+) direction side.

The attachment part opening 65 is formed in parallel to an XZ plane and along the Z direction. Inside the attachment part opening 65 of the optical system block component attachment part 62, the signal cables 54 that connect the ultrasound transducer 50 and the system constituent devices described above are disposed.

In the optical system block component attachment part 62, a pair of guide portions 66 that forms the attachment part opening 65 is formed, and the pair of guide portions 66 extends to the Z(−) direction side along the attachment part opening 65. The optical system block component 80 described below is attached to the pair of guide portions 66 while being slid in the Z direction. With this, the optical system block component 80 is attached to the optical system block component attachment part 62, that is, the ultrasound block component 60, through the pair of guide portions 66.

The channel block component 70 configures the outlet port forming part 34b along with the optical system block component 80, and is formed of a known metal material. The channel block component 70 has the outlet port 52 of the treatment tool that is opened on the Y(+) direction side, and a substantially rectangular opening forming surface 71 parallel to the XZ plane where the outlet port 52 is opened and along the Z direction (including the longitudinal axis 38; the same applies hereinafter). In the present embodiment, description will be provided in connection with a biopsy needle 100 that is used in tissue collection of a lymph node, as an example of the treatment tool.

In both end parts in the X direction of the opening forming surface 71, a pair of flange surfaces 72 parallel to the XZ plane is formed along the Z direction (see FIG. 3). The pair of flange surfaces 72 is used for attachment of the channel block component 70 to the optical system block component 80, and extends outward (X direction) from both end parts in the X direction of the opening forming surface 71.

On a distal end side of the channel block component 70, the locked part 73 that is engaged with the locking part 64 of the ultrasonic attachment part 34a is formed (see FIGS. 3 and 4). The locked part 73 has, for example, an engagement hole with which the locking claw of the locking part 64 is engaged.

As shown in FIG. 4, an in-block pipe line 74 is formed inside the channel block component 70. The in-block pipe line 74 constitutes a pipe line together with the treatment tool insertion channel 23. A distal end side of the in-block pipe line 74 is connected to the outlet port 52, and a proximal end side of the in-block pipe line 74 is connected to the treatment tool insertion channel 23 inserted into the insertion part 12. As a result, a distal end of the biopsy needle 100 inserted from the treatment tool inlet port 24 is guided to the outlet port 52 by way of the treatment tool insertion channel 23 and the in-block pipe line 74, and is led out from the outlet port 52 to the outside.

The optical system block component 80 is formed of a resin material, like the ultrasound block component 60. The optical system block component 80 has a shape corresponding to a divided part on the Y(+) direction side (an upper half side) out of the two divided parts obtained by dividing the outlet port forming part 34b and the body part 34c into two parts in the Y direction (into two parts vertically).

The optical system block component 80 comprises, from a distal end side toward a proximal end side thereof, a pair of channel block component attachment portions 81 that is provided at an interval in the X direction, and an optical system storage portion 82 (see FIG. 3). The pair of channel block component attachment portions 81 and the optical system storage portion 82 are formed integrally.

The pair of channel block component attachment portions 81 extends from positions slightly lower than an apex on the Y(+) direction side of the optical system storage portion 82, that is, positions on the Y(−) direction side with respect to the apex to a distal end side [Z(+) direction side] of the optical system storage portion 82 in a case where the optical system block component 80 is viewed from the X direction side.

A space for attaching the channel block component 70 is secured between the pair of channel block component attachment portions 81. In end parts on the Y(+) direction side of the pair of channel block component attachment portions 81, a pair of planes 81a and a pair of support surfaces 81b are formed (see FIG. 3). The pair of planes 81a has a shape parallel to the XZ plane and along the Z direction.

The pair of support surfaces 81b are surfaces parallel to the pair of planes 81a. The pair of support surfaces 81b are formed at positions that are positions shifted from the pair of planes 81a toward the above-described space and that are positions on the Y(−) direction side lower than the pair of planes 81a as much as a thickness in the Y direction of the pair of flange surfaces 72.

The pair of support surfaces 81b supports the pair of flange surfaces 72 from both sides in the X direction. For this reason, the channel block component 70 is supported to be slidable in the Z direction between the pair of channel block component attachment portions 81 through the pair of flange surfaces 72 and the pair of support surfaces 81b. With this, the channel block component 70 can be attached to the optical system block component 80 while sliding in the Z direction. Then, the channel block component 70 is adhered and fixed to the optical system block component 80.

In a case where the channel block component 70 is attached to the optical system block component 80, the opening forming surface 71 and the pair of planes 81a form a continuous plane 90 (constituting a first surface) (see FIG. 2). The continuous plane 90 is a plane parallel to the XZ plane and along the Z direction, and configures a part of an outer peripheral surface of the distal end hard part 34. In the present embodiment, the continuous plane 90 is a planar surface, but may be a surface having various shapes such as a curved surface, an inclined surface, or a concave-convex surface.

The optical system storage portion 82 has a substantially semi-cylindrical shape, and has a convex surface 84 and a stepped surface 85. The convex surface 84 constitutes the second surface and constitutes a part of an outer peripheral surface of the distal end hard part 34 (optical system storage portion 82). The convex surface 84 is positioned on the Y(+) direction side with respect to the continuous plane 90 and has a shape along the Z direction. The convex surface 84 may be formed in various shapes, such as a curved surface, an inclined surface, or an uneven surface.

The stepped surface 85 is an inclined surface that connects a proximal end side of the continuous plane 90 and a distal end side of the convex surface 84, and configures a part of the outer peripheral surface of the distal end hard part 34. The inclined surface used herein includes a vertical surface having an inclination angle of 90° with respect to the Z direction.

The stepped surface 85 is provided with an observation window 40a of the observation optical system 40 and illumination windows 44a of a pair of illumination optical systems 44.

The observation optical system 40 includes the observation window 40a provided in the stepped surface 85, and a lens system 40b and an imaging element 40c provided in the optical system storage portion 82. The imaging element 40c is a charge-coupled device (CCD) type or complementary metal-oxide-semiconductor (CMOS) type image sensor, and captures an observation image taken in from the observation window 40a via the lens system 40b. Then, the imaging element 40c outputs an imaging signal of the observation image to the system constituent devices through the signal cable 56 inserted into the insertion part 12.

The illumination optical systems 44 are provided on both sides of the observation optical system 40 in the X direction, and each of the illumination optical systems 44 includes the illumination window 44a provided in the stepped surface 85, and the light guide 58 inserted into the insertion part 12. An emission end of the light guide 58 is disposed rearward of each illumination window 44a. With this, illumination light supplied from the system constituent device to each light guide 58 is emitted from each illumination window 44a.

In a case where the channel block component 70 is attached to the optical system block component 80, the pair of guide portions 66 is attached to the optical system block component attachment part 62 of the ultrasound block component 60 through the pair of guide portions 66. In this case, the locking part 64 of the ultrasonic attachment part 34a locks the locked part 73 of the channel block component 70. With this, the movement in the Z direction of the channel block component 70 and the optical system block component 80 with respect to the ultrasound block component 60 is restricted, and the channel block component 70 is assembled to the ultrasound block component 60.

A distal end part of the bendable part 32 is externally fitted on and fixed to proximal end parts of both of the optical system storage portion 82 and the optical system block component attachment part 62 (see FIG. 4). With this, the optical system storage portion 82 and the optical system block component attachment part 62 are held to be not separable in the Y direction by the bendable part 32. As a result, the optical system block component 80 is assembled to the ultrasound block component 60.

As described above, the ultrasound block component 60, the channel block component 70, and the optical system block component 80 are combined, and the distal end hard part 34 is formed. In the distal end hard part 34, the ultrasound transducer 50, the outlet port 52, and the stepped surface 85 (observation window 40a) are disposed in order from the distal end side toward the proximal end side. That is, the outlet port 52 is disposed between the ultrasound transducer 50 and the observation window 40a. For this reason, puncture into a lymph node from a bronchial wall surface by the biopsy needle 100 can be observed through the observation optical system 40.

The distal end hard part 34 of the present embodiment has a shape capable of increasing a contact region of a proximal end part on the Z(−) direction side of the ultrasound transducer 50, that is, a proximal end part on the side of the outlet port 52 from which the biopsy needle 100 is led out, with a bronchial wall surface in acquiring an ultrasound image of a lymph node by the endoscope 1. Hereinafter, the shape will be specifically described.

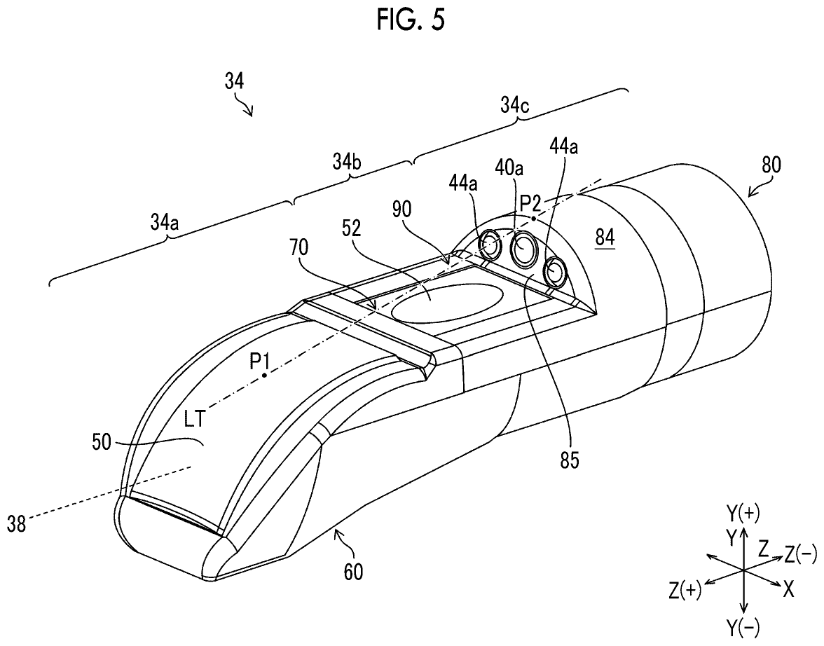

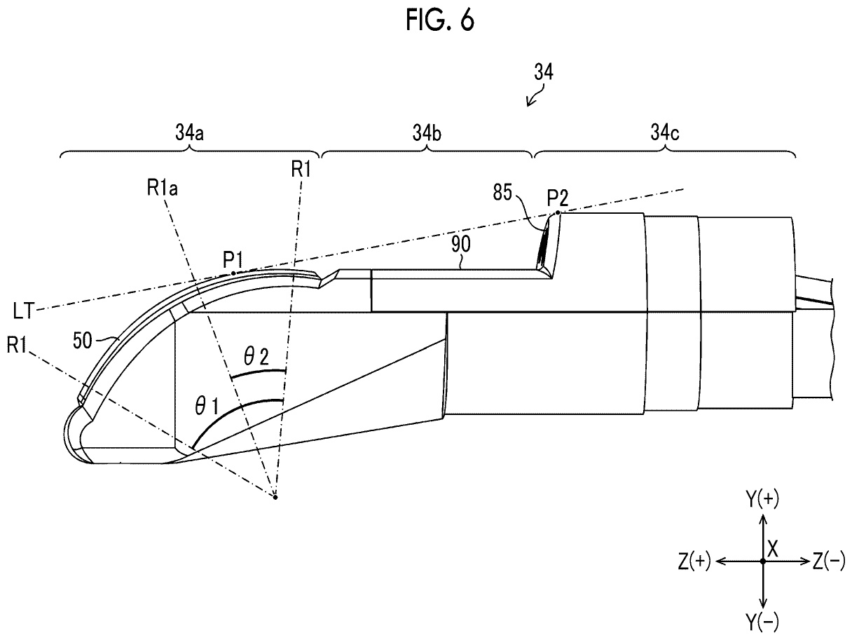

FIG. 5 is a perspective view of the distal end hard part 34 including a tangent line LT. FIG. 6 is a side view of the distal end hard part 34 including the tangent line LT. As shown in FIGS. 5 and 6, in the present embodiment, the shape of the distal end hard part 34 capable of increasing the contact region of the proximal end part of the ultrasound transducer 50 with the bronchial wall surface is defined using the tangent line LT and an effective angle θ1 of the ultrasound transducer 50.

The tangent line LT is a line in contact with the ultrasound transducer 50 and in contact with the stepped surface 85 at a position (apex) closest to the Y(+) direction side. Here, a point where the tangent line LT is in contact with the ultrasound transducer 50 is referred to as a tangent point P1 (constituting a first intersection), and a point where the tangent line LT is in contact with the stepped surface 85 is referred to as a tangent point P2.

The effective angle θ1 of the ultrasound transducer 50 is an angle indicating an irradiation range R1 of an ultrasonic wave emitted from the ultrasound transducer 50 in a case where the distal end hard part 34 is viewed from the X direction side. A one-dot chain line R1a in FIG. 6 indicates a boundary of a range (θ2=⅓×θ1) of ⅓ from a proximal end side [Z(−) direction side] of the effective angle θ1.

In a case where the distal end hard part 34 is viewed from the X direction side as shown in FIG. 6, and in a case where the distal end hard part 34 is brought into contact with a bronchial wall surface, as indicated by the tangent line LT, the tangent point P1 and the tangent point P2 come into contact with the bronchial wall surface. In this case, a region between the tangent point P1 and the tangent point P2 in the distal end hard part 34 is a region that does not easily come into contact with the bronchial wall surface, and conversely, a region on the distal end side with respect to the tangent point P1 is a region that easily comes into contact with the bronchial wall surface. For this reason, the region on the distal end side with respect to the tangent point P1 in the proximal end part of the ultrasound transducer 50 is easily brought into contact with the bronchial wall surface, and conversely, a region on the proximal end side with respect to the tangent point P1 is a region that does not easily come into contact with the bronchial wall surface.

Accordingly, in the present embodiment, the shape of the distal end hard part 34 is adjusted such that the tangent point P1 is included in a range (a range of an angle θ2 in the drawing) of ⅓ on the proximal end side of the effective angle θ1. The adjustment of the shape of the distal end hard part 34 includes adjustment of a shape, an attachment position, and a posture of the ultrasound transducer 50 and adjustment of a shape, a forming position, and an inclination angle of the stepped surface 85. It is preferable that the tangent point P1 is included in a range of ¼ on the proximal end side of the effective angle θ1.

The shape of the distal end hard part 34 is adjusted in this way, whereby, in a case where the distal end hard part 34 is brought into contact with the bronchial wall surface, and in a case where the distal end hard part 34 is viewed from the X direction side, a region between the one-dot chain line R1a and the tangent point P1 in the proximal end part of the ultrasound transducer 50 is reliably brought into contact with the bronchial wall surface (see FIG. 6). That is, a part of the proximal end part of the ultrasound transducer 50 is reliably brought into contact with the bronchial wall surface. As a result, the contact region of the proximal end part of the ultrasound transducer 50 with the bronchial wall surface is increased.

In addition, the shape of the distal end hard part 34 is adjusted such that the position of the tangent point P1 is further deviated to the proximal end side [Z(−) direction side], whereby the contact region of the proximal end part of the ultrasound transducer 50 with the bronchial wall surface is more increased.

As described above, the shape of the distal end hard part 34 is adjusted such that the tangent point P1 is included in the range of ⅓ on the proximal end side of the effective angle θ1 of the ultrasound transducer 50 in a case where the distal end hard part 34 is viewed from the X direction side, whereby the contact region of the proximal end part of the ultrasound transducer 50 with the bronchial wall surface is reliably increased. With this, it is possible to reliably perform visualization of an ultrasound image of a lymph node by the proximal end part of the ultrasound transducer 50.

Next, a modification example of the distal end hard part 34 will be described. In the distal end hard part 34 of the embodiment described above, the ultrasound transducer 50, the outlet port 52, and the stepped surface 85 (observation window 40a) are disposed from the distal end side toward the proximal end side, whereby puncture into the lymph node from the bronchial wall surface by the biopsy needle 100 can be observed with the observation optical system 40.

On the other hand, in the distal end hard part 34 of the modification example, the conditions for reliably observing the puncture of the lymph node with the observation optical system 40 are specifically defined. Since the distal end hard part 34 of the modification example has essentially the same configuration as the distal end hard part 34 of the above-described embodiment, the same reference numerals are assigned to the same functions or configurations as those of the above-described embodiment, and the description thereof will be omitted, and the same effects as those of the above-described embodiment will also be omitted.

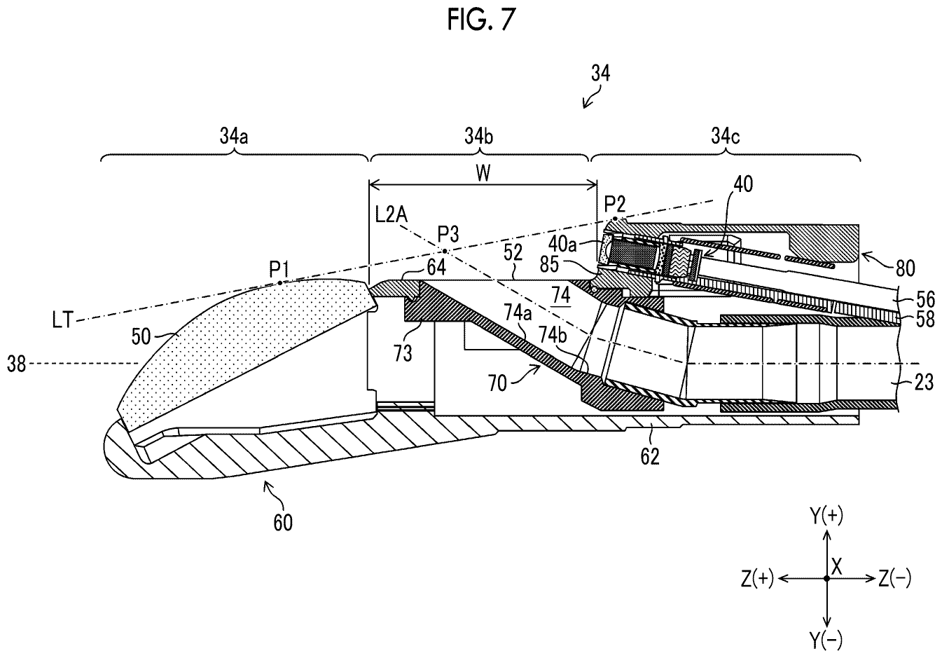

FIG. 7 is a cross-sectional view of the distal end hard part 34 of the modification example. As shown in FIG. 7, the in-block pipe line 74 has a shape bent in two stages in a case where the distal end hard part 34 is viewed from the X direction side. Specifically, the in-block pipe line 74 is configured with a first pipe line 74a and a second pipe line 74b. The first pipe line 74a is connected to the outlet port 52 in the channel block component 70. The second pipe line 74b is provided on the proximal end side of the first pipe line 74a. A distal end part of the treatment tool insertion channel 23 is connected to the second pipe line 74b.

In a case where the distal end hard part 34 is viewed from the X direction side, an intersection P3 (constituting the second intersection) of the tangent line LT and a center line L2A of the first pipe line 74a is positioned within a range W indicating a position on the proximal end side of the distal end hard part 34 with respect to the tangent point P1, and preferably between the ultrasound transducer 50 and the observation window 40a. In a case where the outlet port 52 is opened on the proximal end side of the distal end hard part 34 with respect to the stepped surface 85 (observation window 40a), the intersection P3 is positioned on the proximal end side of the distal end hard part 34 with respect to the tangent point P1.

The intersection P3 corresponds to a position where the biopsy needle 100 led out from the outlet port 52 starts puncture into the lymph node from the bronchial wall surface in a state in which the ultrasound transducer 50 is brought into contact with the bronchial wall surface. The intersection P3 is positioned in the range W, that is, is positioned in front of the observation window 40a, whereby puncture into the lymph node from the bronchial wall surface by the biopsy needle 100 can be reliably observed with the observation optical system 40.

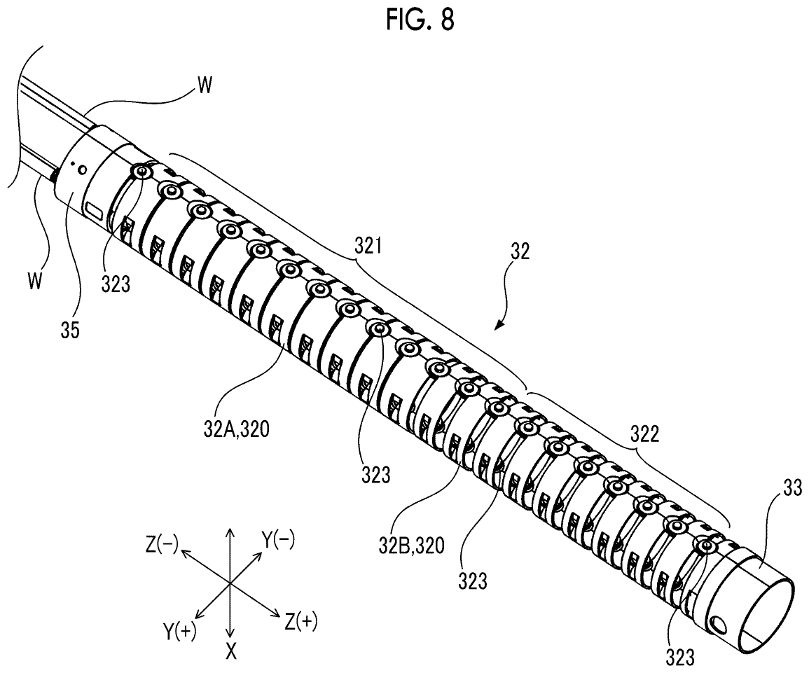

FIG. 8 is a perspective view schematically showing a main part of the bendable part 32 of the endoscope 1 shown in FIG. 1. The bendable part 32 comprises a plurality of movable members 320 arranged in the Z direction, a proximal end-side connecting member 35 that connects the soft part 30 to a most proximal end side (Z(−) direction side) of the plurality of movable members 320, a distal end-side connecting member 33 that connects the distal end hard part 34 to a most distal end side (Z(+) direction side) of the plurality of movable members 320, and two wires W inserted into each of the movable members 320, the proximal end-side connecting member 35, and the distal end-side connecting member 33. Although not shown, the plurality of movable members 320 are covered with a covering member, and an outer skin of the insertion part 12 is provided on the outer periphery thereof.

The bendable part 32 includes a first region 321 and a second region 322 between the first region 321 and the distal end hard part 34. The first region 321 and the second region 322 are configured to be bent with different curvature radii. Specifically, the first region 321 is configured to be bent with a first curvature radius, and the second region 322 is configured to be bent with a second curvature radius less than the first curvature radius. That is, the bendable part 32 is configured to be bent more on the distal end side than on the proximal end side.

In the example of FIG. 8, a total of 19 movable members 320 are provided. The first region 321 is configured by including 12 movable members 320 arranged from the proximal end-side connecting member 35 side among the 19 movable members 320. The second region 322 is configured by including seven movable members 320 arranged from the distal end-side connecting member 33 side among the 19 movable members 320.

Each of the plurality of movable members 320 is formed of a cylindrical member having an axis extending in the Z direction. The plurality of movable members 320 include a first movable member 32A and a second movable member 32B having a width in the Z direction less than that of the first movable member 32A. Among the 19 movable members 320, the first 10 movable members 320 counted from the proximal end-side connecting member 35 side are defined as first movable members 32A, and the movable members 320 counted from the proximal end-side connecting member 35 side, starting from the eleventh onward, are defined as second movable members 32B.

The first region 321 is configured by including ten first movable members 32A and two second movable members 32B. The second region 322 is configured by including seven second movable members 32B. The first movable member 32A closest to the proximal end side of the first region 321 is rotationally movably connected to the tubular proximal end-side connecting member 35. The second movable member 32B closest to the distal end side in the second region 322 is rotationally movably connected to the tubular distal end-side connecting member 33.

Each of the two wires W passes through the soft part 30 and is connected to the angle lever 16. Each movable member 320, the proximal end-side connecting member 35, and the distal end-side connecting member 33 are made of, for example, metal, resin, or the like. The distal end of each wire W is anchored to the distal end-side connecting member 33.

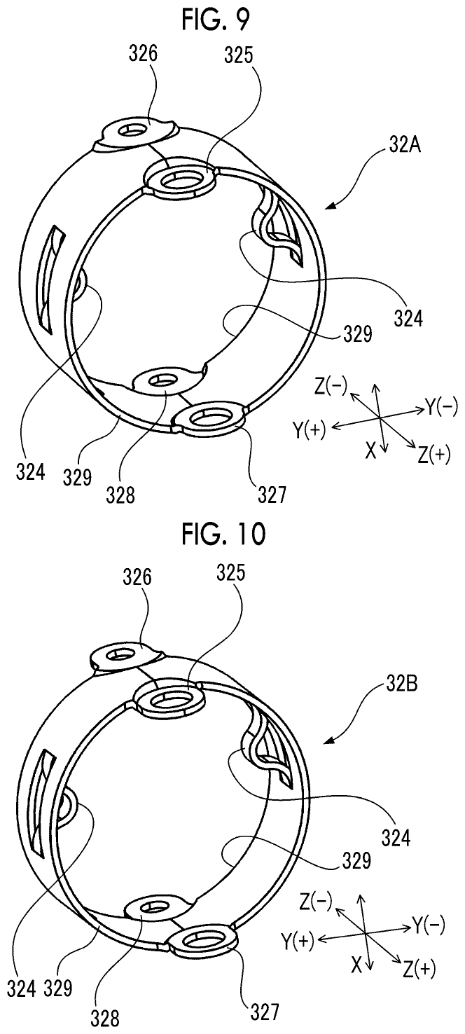

FIG. 9 is an enlarged perspective view of the first movable member 32A shown in FIG. 8. FIG. 10 is an enlarged perspective view of the second movable member 32B shown in FIG. 8.

An end surface 329 of the first movable member 32A on the Z(+) direction side and an end surface 329 of the first movable member 32A on the Z(−) direction side are each a flat surface perpendicular to the Z direction.

The first movable member 32A is provided with a connection piece 325 that protrudes from an end surface 329 on the Z(+) direction side to the Z(+) direction at one end part in the X direction, and a connection piece 326 that protrudes from an end surface 329 on the Z(−) direction side to the Z(−) direction at the one end part. The connection piece 325 and the connection piece 326 have a plate shape having a thickness in the X direction. Each of the connection piece 325 and the connection piece 326 is provided with a connecting hole that penetrates in the X direction.

The first movable member 32A is provided with a connection piece 327 that protrudes from an end surface 329 on the Z(+) direction side to the Z(+) direction at the other end part in the X direction, and a connection piece 328 that protrudes from an end surface 329 on the Z(−) direction side to the Z(−) direction at the other end part in the X direction. The connection piece 327 and the connection piece 328 have a plate shape having a thickness in the X direction. Each of the connection piece 327 and the connection piece 328 is provided with a connecting hole that penetrates in the X direction.

A pair of wire guides 324 into which two wires W are inserted is provided on an inner peripheral surface of the first movable member 32A in the Y direction so as to face each other between one end part and the other end part in the X direction.

As shown in FIG. 10, the second movable member 32B has the same configuration as the first movable member 32A, except that the width of the second movable member 32B in the Z direction (the distance between the end surface 329 on the Z(+) direction side and the end surface 329 on the Z(−) direction side) is less than the width of the first movable member 32A.

As shown in FIG. 8, the connection piece 325 of each movable member 320 excluding two movable members 320 closest to the proximal end side and the distal end side among all the movable members 320 is rotationally movably connected to the connection piece 326 of the movable member 320 adjacent to the distal end side by the connecting member 323, such as a rivet. The connection piece 326 of each movable member 320 is rotationally movably connected to the connection piece 325 of the movable member 320 adjacent to the proximal end side thereof by a connecting member, such as a rivet. The connection piece 327 of each movable member 320 is rotationally movably connected to the connection piece 328 of the movable member 320 adjacent to the distal end side thereof by a connecting member, such as a rivet. The connection piece 328 of each movable member 320 is rotationally movably connected to the connection piece 327 of the movable member 320 adjacent to the proximal end side thereof by a connecting member, such as a rivet.

The movable member 320 closest to the proximal end side among all the movable members 320 is rotatably connected to the proximal end-side connecting member 35 by a connecting member 323, such as a rivet, at the connection piece 326 and the connection piece 328. The movable member 320 closest to the distal end side among all the movable members 320 is rotatably connected to the distal end-side connecting member 33 by a connecting member 323, such as a rivet, at the connection piece 325 and the connection piece 327.

In the endoscope 1, the wire W inserted into the wire guide 324 on the Y(+) direction side of the wire guide 324 of the movable member 320 is pulled by the rotation operation of the angle lever 16 in the A1 direction, so that the bendable part 32 is bent in the Y(+) direction (A2 direction in FIG. 1). The wire W inserted into the wire guide 324 on the Y(−) direction side of the wire guide 324 of the movable member 320 is pulled by the rotation operation of the angle lever 16 in the −A1 direction, so that the bendable part 32 is bent in the Y(−) direction (the −A2 direction in FIG. 1).

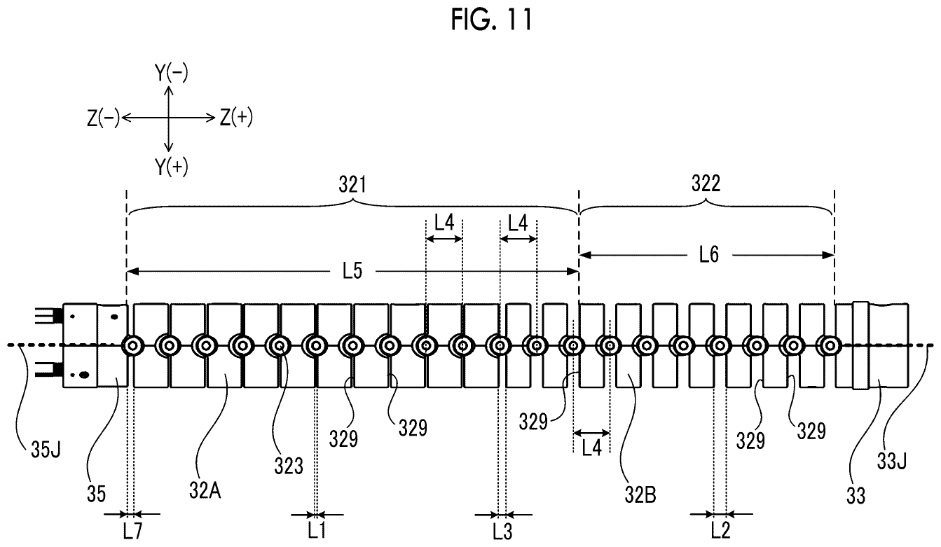

FIG. 11 is a view schematically showing a state in which the bendable part 32 shown in FIG. 8 is viewed in the direction X. As shown in FIG. 11, a gap is provided between the end surfaces 329 of two adjacent first movable members 32A, and a length of the gap in the Z direction is a distance L1. A gap is provided between the end surfaces 329 of the two adjacent second movable members 32B, and a length of the gap in the Z direction is a distance L2. A gap is provided between the end surfaces 329 of the first movable member 32A that is the tenth movable member counted from the proximal end side and the second movable member 32B adjacent to the first movable member 32A, and a length of the gap in the Z direction is a distance L3. Intervals L4 in the Z direction between the connecting members 323 that connect the adjacent movable members 320 to be rotationally movable are uniform as a whole.

Since the interval L4 is uniform, the distance L2 is larger than the distance L1 due to a difference in width between the first movable member 32A and the second movable member 32B in the Z direction. The distance L3 has a magnitude obtained by adding half of the distance L2 and half of the distance L1.

A length of a gap between the proximal end-side connecting member 35 and the first movable member 32A adjacent to the proximal end-side connecting member 35 in the Z direction is a distance L7. It is preferable that the distance L7 is larger than the distance L1. Further, it is preferable that the distance L7 is less than the distance L2. It is preferable that the distance L7 is, for example, the same as the distance L3 or less than the distance L3. In this way, it is easy to increase the bending angle in a case where the first region 321 is maximally bent while suppressing the length of the first region 321 in the Z direction.

As shown in FIG. 11, a portion from a distal end-side end surface of the proximal end-side connecting member 35 to a proximal end-side end surface 329 of the second movable member 32B that is the seventh counted from the distal end-side connecting member 33 side constitutes the first region 321. In addition, a portion from the proximal end-side end surface of the distal end-side connecting member 33 to the proximal end-side end surface 329 of the second movable member 32B that is the seventh counted from the distal end-side connecting member 33 side constitutes the second region 322. It is preferable that a length L5 of the first region 321 in the Z direction is equal to or greater than a length L6 of the second region 322 in the Z direction.

In a case where the rotation angle of the angle lever 16 is 0°, as shown in FIG. 11, an axis 35J of the proximal end-side connecting member 35 and an axis 33J of the distal end-side connecting member 33 are arranged in a linear shape, and an angle formed by the axis 35J and the axis 33J is 0°. The axis 33J coincides with the axis of the distal end hard part 34. The axis 35J coincides with the axis of the soft part 30. In a case where the angle lever 16 is rotationally operated, the axis 33J of the distal end-side connecting member 33 is inclined with respect to the axis 35J of the proximal end-side connecting member 35, and an angle formed by the axis 35J and the axis 33J increases. An angle formed by the axis 35J and the axis 33J is defined as a bending angle of the bendable part 32. In the state shown in FIG. 11, the bending angle is 0°.

The bending angle of the bendable part 32 (a second upper limit value of the bending angle in a case of being rotated in the A1 direction) in a case where the angle lever 16 is rotated in the A1 direction to the maximum extent (in a case of a rotation angle of 45°) is also referred to as the maximum bending angle in the A2 direction. In consideration of easily and appropriately bringing the surface of the distal end hard part 34 on the ultrasound transducer 50 side into contact with a desired site in the bronchus, the maximum bending angle in the A2 direction is preferably in a range of 165° or more and 195° or less, and more preferably 180°.

The bending angle of the bendable part 32 in a case where the angle lever 16 is rotated in the −A1 direction to the maximum extent (a third upper limit value of the bending angle in a case where the bendable part 32 is rotated in the −A1 direction) is also referred to as the maximum bending angle in the −A2 direction. It is preferable that the maximum bending angle in the −A2 direction is less than the maximum bending angle in the A2 direction in consideration of the followability to the shape of the bronchus, the operability, and the optimization of the manufacturing cost. The maximum bending angle in the −A2 direction is, for example, preferably in a range of 75° or more and 115° or less, and more preferably 90°.

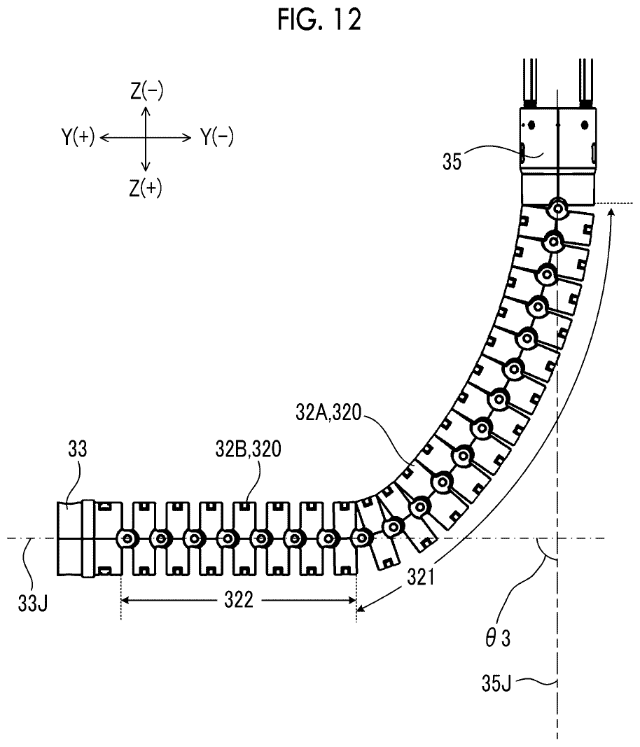

FIG. 12 is a schematic view for describing a bent state of the bendable part 32 in a case where the angle lever 16 is rotated in the A1 direction from the state shown in FIG. 11. The Y direction and the Z direction shown in FIG. 12 are shown as directions in a state in which the bendable part 32 is linearly extended up and down in the drawing (a state in which the bendable part 32 is not bent).

In a case where the angle lever 16 is rotationally operated in the A1 direction in the first range (range of a rotation angle greater than 0° and 250 or less), as shown in FIG. 12, only the first region 321 out of the first region 321 and the second region 322 is bent in the Y(+) direction. On the other hand, the second region 322 maintains a state of extending along the axis 33J (synonymous with the axis of the distal end hard part 34).

FIG. 12 shows a state in which the angle lever 16 is rotationally operated in the first range and the bending angle is at its maximum (angle θ3) (state in which the rotation angle 250 of the angle lever 16 is obtained). Until the angle lever 16 reaches the rotation angle of 25°, the gap between the proximal end-side connecting member 35 and the adjacent movable member 320, the gap between the movable members 320 included in the first region 321, and the gap between the movable member 320 on the most distal end side in the first region 321 and the adjacent movable member 320 on the distal end side change in a direction in which the gaps gradually narrow, and the first region 321 is bent in the Y(+) direction.

On the other hand, while the first region 321 is bent, the gap between the movable members 320 included in the second region 322 and the gap between the distal end-side connecting member 33 and the adjacent movable member 320 are maintained in the state shown in FIG. 11. The angle θ3 shown in FIG. 12 is 90° as an example. As described above, until the bending angle reaches 0° to 90°, the orientation of the distal end hard part 34 can be changed while the second region 322 is kept in a linear shape.

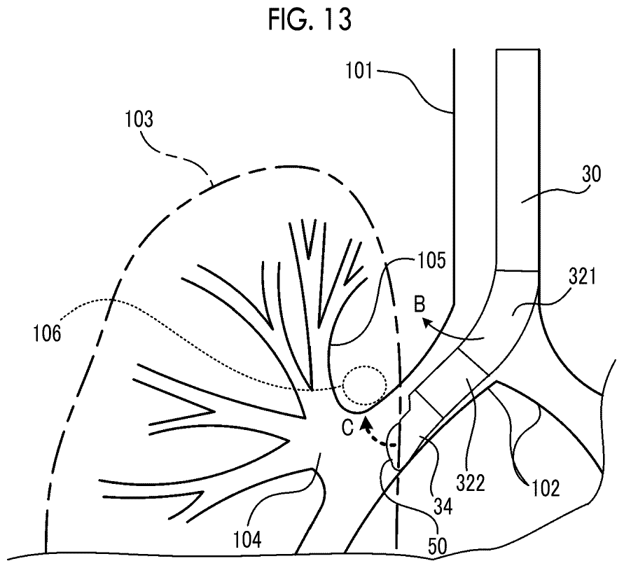

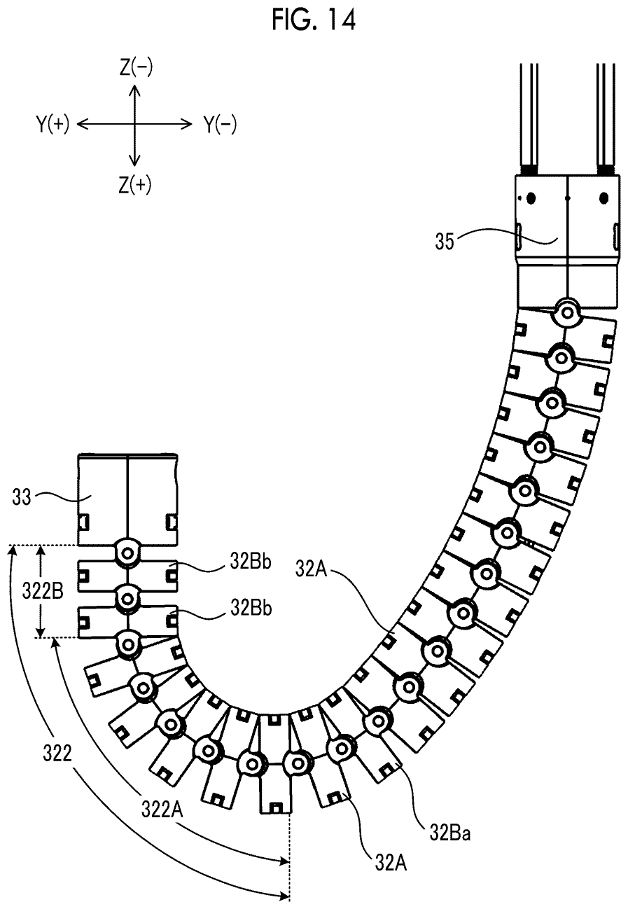

FIG. 13 is a schematic view for describing a state in which the endoscope 1 is inserted into the bronchus and used. FIG. 13 shows a trachea 101 of the subject, a lung 103 of the subject, a pair of main bronchi 102 that branch off from the trachea 101 to the left and right and to the lower side, and a bronchus 105 that branches off from the main bronchus 102 to the upper side via a superior lobar bronchus 104. In the clinical field, the treatment of a same-side pulmonary hilar lymph node 106 on the upper side of the back of the main bronchus 102 may be performed.

As described above, the endoscope 1 can bend the first region 321 with a large curvature radius while holding the second region 322 in a linear shape. As shown in FIG. 13, by bending the first region 321, the distal end hard part 34 can be smoothly inserted along a gentle and relatively long bending path from the trachea 101 to the main bronchus 102.

In a case where the first region 321 is further bent in a B direction (corresponding to the Y(+) direction) in the drawing in the state shown in FIG. 13, the distal end hard part 34 moves in a C direction in the drawing. Since the second region 322 is held in a linear shape, the tangent point P1 (see FIG. 5) of the distal end hard part 34 can be moved without significantly changing the relative position with respect to the same-side pulmonary hilar lymph node 106. Therefore, it is easy to bring the vicinity of the tangent point P1 of the distal end hard part 34 into contact with the same-side pulmonary hilar lymph node 106.

For example, as in the related art, a configuration is assumed in which only the distal end side is bent while the proximal end side maintains a linear shape in the bendable part 32. In this configuration, it is difficult to insert the distal end hard part 34 to the position of the same-side pulmonary hilar lymph node 106 deep in the main bronchus 102.

In addition, a configuration is assumed in which the bendable part 32 is bent as a whole in accordance with the rotation operation of the angle lever 16. In this configuration, there is a possibility that the ultrasound transducer 50 is directed toward the proximal end side in the process of inserting the distal end hard part 34 to the depth of the main bronchus 102, and it is difficult to dispose the vicinity of the tangent point P1 to face the same-side pulmonary hilar lymph node 106.

According to the endoscope 1 of the present embodiment, the vicinity of the tangent point P1 can be easily disposed to face the same-side pulmonary hilar lymph node 106 located deep and on the upper side of the main bronchus 102. Therefore, in a case in which the treatment is performed while observing the same-side pulmonary hilar lymph node 106 with the ultrasound image, it is possible to perform the treatment with high accuracy.

In order to bring the ultrasound transducer 50 into contact with the wall surface of the main bronchus 102 in the vicinity of the same-side pulmonary hilar lymph node 106, it is sufficient that the bending angle of the bendable part 32 in a state in which the second region 322 is held in a linear shape is about 50° to 65°. That is, a first upper limit value (angle θ3 shown in FIG. 12) of the bending angle of the bendable part 32 in a state in which the second region 322 is held in a linear shape (state in which the angle lever 16 is rotated and operated in the first range) may be set in a range of at least 50° or more and 65° or less.

However, in a case where a treatment such as a biopsy is performed on the same-side pulmonary hilar lymph node 106, the biopsy needle 100 is inserted into the same-side pulmonary hilar lymph node 106 in a state in which the ultrasound transducer 50 is in contact with the wall surface of the main bronchus 102. During the puncture by the biopsy needle 100, the first region 321 may be pushed back by about 30° in a direction opposite to the B direction in FIG. 13, and in this case, there is a possibility that the ultrasound transducer 50 is separated from the wall surface of the main bronchus 102.

Therefore, the first upper limit value is preferably set to be larger than a range of 50° or more and 65° or less. In this manner, the biopsy needle 100 can be inserted while further bending the first region 321 in the B direction in the state shown in FIG. 13 (for example, a state in which the bending angle is 60°). In this way, it is possible to puncture the same-side pulmonary hilar lymph node 106 with the biopsy needle 100 while preventing the ultrasound transducer 50 from being separated from the wall surface of the main bronchus 102.

From the viewpoint of appropriately performing the observation and the treatment of the same-side pulmonary hilar lymph node 106, the above-described first upper limit value is preferably 50° or more and 100° or less, more preferably 60° or more and 95° or less, and still more preferably 90°.

FIG. 14 is a schematic view for describing a bent state of the bendable part 32 in a case where the rotation angle of the angle lever 16 is increased from the state shown in FIG. 12. In a case where the angle lever 16 is rotationally operated in a second range (a range of a rotation angle greater than 25° and 45° or less) exceeding the first range in the A1 direction, only the second region 322 out of the first region 321 and the second region 322 is bent as shown in FIG. 14. On the other hand, the first region 321 maintains the bent state shown in FIG. 12 (the bent state in a case where the rotation angle is 25° (the rotation angle of the end on the second range side in the first range)).

FIG. 14 shows a state in which the angle lever 16 is rotationally operated in the second range and the bending angle is at its maximum (180° in the example of the drawing) (a state in which the rotation angle of the angle lever 16 is 45°). Until the angle lever 16 rotates from the rotation angle of 25° to the rotation angle of 45°, the gap between five movable members 320 excluding two movable members 320 on the distal end side (denoted by reference numeral 32Bb in FIG. 14) of the movable members 320 included in the second region 322 changes in a direction in which the gap gradually decreases, and a portion 322A (a part closer to the proximal end side with respect to the two movable members 32Bb) of the second region 322 is bent with a smaller curvature radius than the first region 321. As shown in FIG. 14, in a state where the rotation angle of the angle lever 16 is 45°, the axes of the two movable members 32Bb coincide with the axis 33J of the distal end-side connecting member 33. That is, even in a case where the angle lever 16 is rotated to the maximum extent in the A1 direction, the remaining portion 322B excluding the portion 322A in the second region 322 maintains a linear shape.

As described above, the other portion 322B of the second region 322 is configured such that the bending operation by the rotation of the angle lever 16 is not possible. In the present embodiment, the other portion 322B is prevented from being bent by regulating the rotatable range of the angle lever 16. However, the remaining portion 322B can be bent in each of the Y(+) direction and the Y(−) direction with the same curvature radius as the portion 322A in a case where an external force is applied. Since the other portion 322B is linear, the lead-out of the biopsy needle 100 from the outlet port 52 can be smoothly performed in a case where the lead-out is performed. In the second region 322, the other portion 322B is not essential and can be omitted.

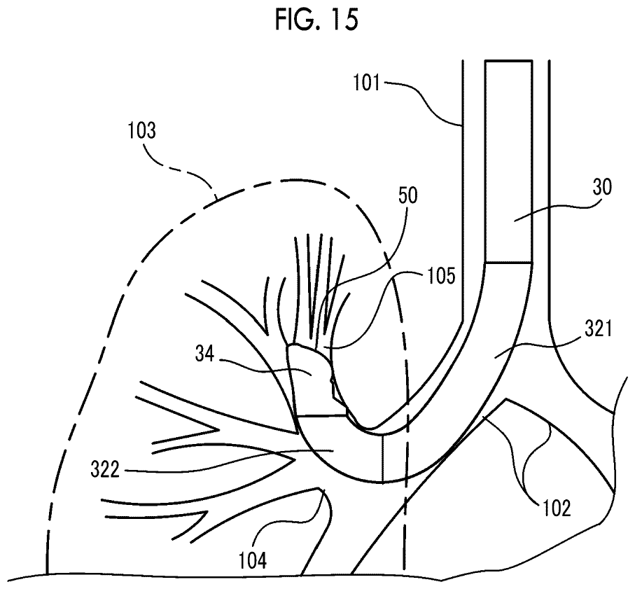

FIG. 15 is a schematic view for describing a state in which the endoscope 1 is inserted into the bronchus and used. In the clinical field, it may be necessary to observe or treat the periphery of the bronchus 105 that branches from the superior lobar bronchus 104 to the upper side.

In a case where the distal end hard part 34 is inserted into the bronchus 105, the distal end hard part 34 can be smoothly inserted along a gentle and relatively long bending path from the trachea 101 to the superior lobar bronchus 104 while bending the first region 321. Further, since the second region 322 can be bent with a small curvature radius from a state in which the first region 321 is maximally bent, as shown in FIG. 15, the distal end hard part 34 can be easily inserted into the back of the bronchus 105 that branches at an acute angle with respect to the path from the trachea 101 to the superior lobar bronchus 104.

Even in a case where the distal end hard part 34 is inserted into the thin bronchus, the remaining portions 322B of the second region 322 can be passively bent, so that the insertion can be smoothly performed.

In addition, in a case where the second region 322 is bent from a state in which the first region 321 is bent to the maximum extent, the bent state of the first region 321 is maintained. Therefore, the direction of the distal end hard part 34 can be changed by bending the second region 322 in a state where the first region 321, for example, is brought into contact with the wall surface of the main bronchus 102 and is stabilized.

As described above, in order to smoothly insert the distal end hard part 34 along the path from the trachea 101 through the main bronchus 102 to the superior lobar bronchus 104, and from the superior lobar bronchus 104 to the bronchus 105 on the upper side, the relationship between the length L5 of the first region 321 and the length L6 of the second region 322 shown in FIG. 11 is important.

In a case where the length L6 is larger than the length L5, it is not easy to handle the distal end hard part 34 in a narrow space from the superior lobar bronchus 104. On the other hand, by making the length L6 and the length L5 the same, the distal end hard part 34 can be inserted into the depth of the bronchus 105 while facilitating the handling of the distal end hard part 34 in a narrow space beyond the superior lobar bronchus 104.

In addition, in a case where the length L6 is set to be less than the length L5, it is possible to further facilitate the handling of the distal end hard part 34 in a narrow space beyond the superior lobar bronchus 104. However, in a case where the length L6 is excessively small, it is not easy to insert the distal end hard part 34 into the depth of the bronchus 105. In addition, in a case where the length L6 is excessively small, it is not easy to increase the curvature of the second region 322.

In addition, as shown in FIG. 13, in a case where the distal end hard part 34 approaches the same-side pulmonary hilar lymph node 106, it is difficult to perform operation of moving the distal end hard part 34 to the back of the main bronchus 102 in a case where the length L6 is excessively increased. In addition, in a case where the length L6 is excessively small, for example, in a state in which the proximal end of the first region 321 is inserted into the main bronchus 102, it is necessary to bend the distal end hard part 34 in a direction in which the distal end hard part 34 is brought close to the same-side pulmonary hilar lymph node 106, and as shown in FIG. 13, in a state in which the proximal end of the first region 321 is supported and stabilized by the wall surface of the trachea 101, it is difficult to perform operation of bending the first region 321 and bringing the distal end hard part 34 close to the same-side pulmonary hilar lymph node 106.

As a result of repeated verification in consideration of the above circumstances, it was found that, by setting the length L6 to 0.5 times or more and 1.0 times or less the length L5, it is possible to improve the operability and the treatment accuracy in a case where the distal end hard part 34 approaches the same-side pulmonary hilar lymph node 106, and it is possible to improve the operability of the insertion while the distal end hard part 34 is inserted into the bronchus 105.

It is preferable that the length L5 is set to 34.5 mm, the length L6 is set to 19.6 mm, the distance L1 is set to 0.2 mm, the distance L2 is set to 0.95 mm, the distance L3 is set to 0.575 mm, the interval L4 is set to 2.8 mm, the distance L7 is set to 0.5 mm, the outer diameter of the movable member 320 is set to 6.3 mm, and a curvature R of the second region 322 is set to R6.

As described above, in a case where the examination or treatment of the bronchus is performed, it is preferable that the second upper limit value of the bending angle of the bendable part 32 in a case where the angle lever 16 is operated in the second range is set to 165° or more and 195° or less. In this manner, as described above, the distal end hard part 34 can appropriately approach the bronchus that branches from the superior lobar bronchus 104 at an acute angle.

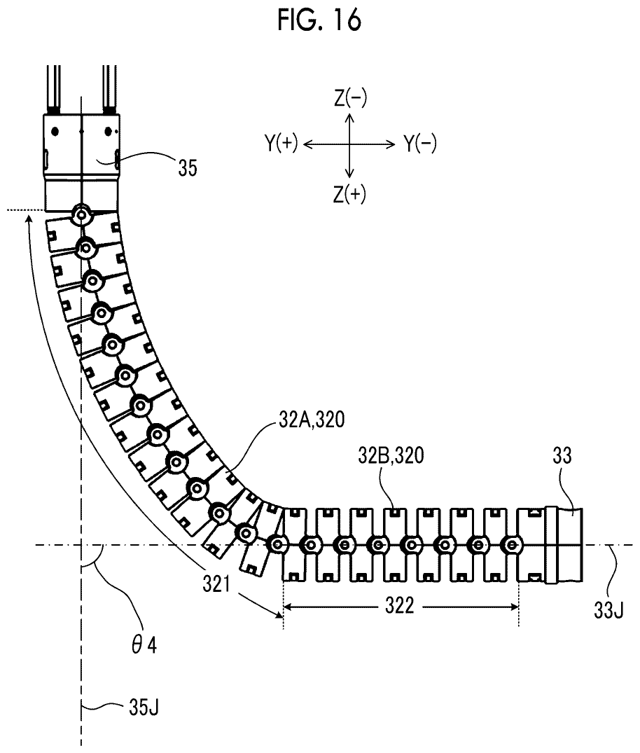

FIG. 16 is a schematic view for describing a bent state of the bendable part 32 in a case where the angle lever 16 is rotated in the −A1 direction from the state shown in FIG. 11. The Y direction and the Z direction shown in FIG. 16 are shown as directions in a state in which the bendable part 32 is linearly extended up and down in the drawing (a state in which the bendable part 32 is not bent).

In a case where the angle lever 16 is rotationally operated in a third range (a range of a rotation angle greater than 0° and 25° or less) in the −A1 direction, as shown in FIG. 16, only the first region 321 out of the first region 321 and the second region 322 is bent in the Y(−) direction. On the other hand, the second region 322 maintains a state of extending along the axis 33J (synonymous with the axis of the distal end hard part 34).

The bend state of the bendable part 32 in a case where the angle lever 16 is rotationally operated in the third range in the −A1 direction is the same as that in a case where the angle lever 16 is rotationally operated in the A1 direction in the first range, except that the bending direction of the bendable part 32 is the Y(−) direction. FIG. 16 shows a state in which the angle lever 16 is rotationally operated in the third range to have a maximum bending angle (angle θ4). The angle θ4 is 90° as an example. The angle θ4 is preferably the same as the angle θ3, but may be less than the angle θ3.

In the present embodiment, the angle lever 16 can be rotated only in the third range with respect to the −A1 direction. That is, the third upper limit value (angle θ4) of the bending angle of the bendable part 32 in the Y(−) direction in a case where the angle lever 16 is rotationally operated is less than the second upper limit value.

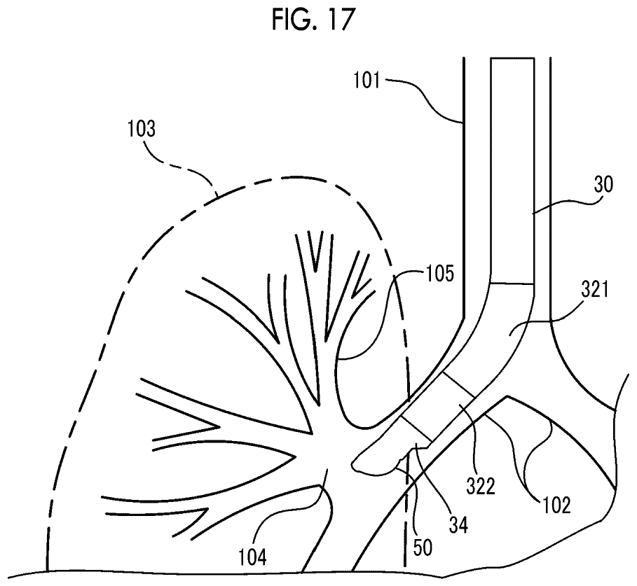

As shown in FIG. 17, in a case where the distal end hard part 34 is assumed to be inserted into a bronchus on a lower side than the superior lobar bronchus 104, the maximum bending angle in the Y(−) direction may be less than the maximum bending angle in the Y(+) direction. In this way, by configuring the bending in the Y(−) direction as necessary and sufficient, it is possible to perform efficient operation depending on the part to be observed. In particular, in a case where the distal end hard part 34 is inserted into a narrow region such as a bronchus, it is preferable to bend the distal end hard part 34 as much as necessary, and from this viewpoint, it is preferable that the maximum bending angle in the Y(−) direction is less than the maximum bending angle in the Y(+) direction.

As described above, at least the following matters are described in the present specification.

(1)

An endoscope including:
an insertion part including a distal end part that includes an ultrasound transducer array, and a bendable part that is provided on a proximal end side of the distal end part; and
a bending operation part that is capable of performing a bending operation on the bendable part,
in which the bendable part includes a first region, and a second region between the first region and the distal end part,
the first region is bent toward a surface side of the distal end part on which the ultrasound transducer array is provided in a case where the bending operation part is operated in a first range, and
the second region is held in a state of extending along an axis of the distal end part in a case where the bending operation part is operated in the first range.
   (2)

The endoscope according to (1),
in which the distal end part has an outlet port of a treatment tool positioned closer to the bendable part than the ultrasound transducer array.
   (3)

The endoscope according to (2),
in which a first upper limit value of a bending angle of the bendable part in a case where the bending operation part is operated in the first range is 50° or more and 100° or less.
   (4)

The endoscope according to (3),
in which the first upper limit value is 60° or more and 95° or less.
   (5)

The endoscope according to any one of (1) to (4),
in which, in a case where the bending operation part is operated in a second range exceeding the first range, at least a part of the second region is bent toward the surface side of the distal end part on which the ultrasound transducer array is provided.
   (6)

The endoscope according to (5),
in which, in a case where the bending operation part is operated in the second range, a bent state of the first region at a time when the bending operation part is operated to an end of the first range on a side of the second range is maintained.
   (7)

The endoscope according to (6),
in which an end part of the second region on a distal end part side is configured not to be subjected to the bending operation by the bending operation part.
   (8)

The endoscope according to (7),
in which the end part of the second region on the distal end part side is configured to be bent by an external force.
   (9)

The endoscope according to any one of (5) to (8),
in which a second upper limit value of a bending angle of the bendable part in a case where the bending operation part is operated in the second range is 165° or more and 195° or less.
   (10)

The endoscope according to (9),
in which the bendable part is further bendable to a side opposite to the surface side of the distal end part on which the ultrasound transducer array is provided, and
a third upper limit value of the bending angle to the opposite side in a case where the bending operation part is operated is smaller than the second upper limit value.
   (11)

The endoscope according to (10),
in which the third upper limit value is equal to or less than an upper limit value of the bending angle of the bendable part in a case where the bending operation part is operated in the first range.
   (12)

The endoscope according to any one of (1) to (11),
in which a length of the second region in a longitudinal axis direction of the insertion part is equal to or less than a length of the first region in the longitudinal axis direction.
   (13)

The endoscope according to (12),
in which the length of the second region in the longitudinal axis direction is 0.5 times or more the length of the first region in the longitudinal axis direction.
   (14)

The endoscope according to any one of (1) to (13),
in which an outer peripheral surface of the distal end part includes
   a first surface provided on a proximal end side of the ultrasound transducer array and extending along a longitudinal axis of the insertion part,
   a second surface provided on a proximal end side of the first surface, extending along the longitudinal axis, and positioned on one side in a first direction perpendicular to the longitudinal axis with respect to the first surface, and
   a stepped surface connecting the proximal end side of the first surface and a distal end side of the second surface, and
in a case where a direction perpendicular to both the longitudinal axis and the first direction is defined as a second direction, an angle indicating an irradiation range of an ultrasonic wave emitted from the ultrasound transducer array in a case where the distal end part is viewed from a second direction side is defined as an effective angle, and an intersection between a tangent line, that is in contact with the ultrasound transducer array and in contact with the stepped surface at a position closest to the one side, and the ultrasound transducer array is defined as a first intersection, the first intersection is included within a range of ⅓ of the effective angle on the proximal end side.
   (15)

The endoscope according to (14), further including:
an outlet port that is provided on the outer peripheral surface of the distal end part and that is open on the one side, and through which a treatment tool is led out;
a pipe line that is connected to the outlet port in the distal end part and through which the treatment tool is inserted; and
an observation window of an observation optical system that is provided on the stepped surface,
in which, in a case where the distal end part is viewed from the second direction side, a second intersection, which is an intersection between a center line of a distal end part of the pipe line connected to the outlet port and the tangent line, is positioned closer to the proximal end side of the distal end part than the first intersection.
   (16)

The endoscope according to (15),
in which the second intersection is positioned between the ultrasound transducer array and the stepped surface.

### EXPLANATION OF REFERENCES

1: ultrasonic endoscope
10: operating part
12: insertion part
14: universal cord
16: angle lever
22: suction button
23: treatment tool insertion channel
24: treatment tool inlet port
30: soft part
32: bendable part
32A: first movable member
32B, 32Bb: second movable member
320: movable member
33: distal end-side connecting member
33J, 35J: axis
34: distal end hard part
34a: ultrasonic attachment part
34b: outlet port forming part
34c: body part
35: proximal end-side connecting member
38: longitudinal axis
40: observation optical system
40a: observation window
40b: lens system
40c: imaging element
44: illumination optical system
44a: illumination window
50: ultrasound transducer
52: outlet port
54, 56: signal cable
58: light guide
60: ultrasound block component
62: optical system block component attachment part
64: locking part
65: attachment part opening
66: guide portion
70: channel block component
71: opening forming surface
72: flange surface
73: locked part
74: in-block pipe line
74a: first pipe line
74b: second pipe line
80: optical system block component
81: channel block component attachment portion
81a: plane
81b: support surface
82: optical system storage portion
84: convex surface
85: stepped surface
90: continuous plane
100: biopsy needle
101: trachea
102: main bronchus
103: lung
104: superior lobar bronchus
105: bronchus
106: same-side pulmonary hilar lymph node
321: first region
322: second region
322A: portion
322B: remaining portion
323: connecting member
324: wire guide
325, 326, 327, 328: connection piece
329: end surface
P1, P2: tangent point
P3: intersection
θ1: effective angle
R1: irradiation range
L2A: center line
L1, L2, L3, L7: distance
L4: interval
L5, L6: length

What is claimed is:

1. An endoscope comprising:
an insertion part including a distal end part that includes an ultrasound transducer array, and a bendable part that is provided on a proximal end side of the distal end part; and
a bending operation part that is capable of performing a bending operation on the bendable part,
wherein the bendable part includes a first region, and a second region between the first region and the distal end part,
the first region is bent toward a side of a surface of the distal end part on which the ultrasound transducer array is provided in a case where the bending operation part is operated in a first range, and
the second region is held in a state of extending along an axis of the distal end part in a case where the bending operation part is operated in the first range.

2. The endoscope according to claim 1,
wherein the distal end part has an outlet port of a treatment tool positioned closer to the bendable part than the ultrasound transducer array.

3. The endoscope according to claim 2,
wherein a first upper limit value of a bending angle of the bendable part in a case where the bending operation part is operated in the first range is 50° or more and 100° or less.

4. The endoscope according to claim 3,
wherein the first upper limit value is 60° or more and 95° or less.

5. The endoscope according to claim 1,
wherein, in a case where the bending operation part is operated in a second range exceeding the first range, at least a part of the second region is bent toward the side of the surface of the distal end part on which the ultrasound transducer array is provided.

6. The endoscope according to claim 5,
wherein, in a case where the bending operation part is operated in the second range, a bent state of the first region at a time when the bending operation part is operated to an end of the first range on a side of the second range is maintained.

7. The endoscope according to claim 6,
wherein an end part of the second region on a side of the distal end part is configured not to be subjected to the bending operation by the bending operation part.

8. The endoscope according to claim 7,
wherein the end part of the second region on the side of the distal end part is configured to be bendable by an external force.

9. The endoscope according to claim 8,
wherein a second upper limit value of a bending angle of the bendable part in a case where the bending operation part is operated in the second range is 165° or more and 195° or less.

10. The endoscope according to claim 9,
wherein the bendable part is further bendable to an opposite side to the side of the surface of the distal end part on which the ultrasound transducer array is provided, and
a third upper limit value of the bending angle to the opposite side in a case where the bending operation part is operated is smaller than the second upper limit value.

11. The endoscope according to claim 10,
wherein the third upper limit value is equal to or less than an upper limit value of the bending angle of the bendable part in a case where the bending operation part is operated in the first range.

12. The endoscope according to claim 11,
wherein a length of the second region in a direction of a longitudinal axis of the insertion part is equal to or less than a length of the first region in the direction of the longitudinal axis.

13. The endoscope according to claim 12,
wherein the length of the second region in the direction of the longitudinal axis is 0.5 times or more the length of the first region in the direction of the longitudinal axis.

14. The endoscope according to claim 13,
wherein an outer peripheral surface of the distal end part includes
a first surface provided on a proximal end side of the ultrasound transducer array and extending along the longitudinal axis of the insertion part,
a second surface provided on a proximal end side of the first surface, extending along the longitudinal axis, and positioned on one side in a first direction perpendicular to the longitudinal axis with respect to the first surface, and
a stepped surface connecting the proximal end side of the first surface and a distal end side of the second surface, and
in a case where a direction perpendicular to both the longitudinal axis and the first direction is defined as a second direction, an angle indicating an irradiation range of an ultrasonic wave emitted from the ultrasound transducer array in a case where the distal end part is viewed from a side of the second direction is defined as an effective angle, and an intersection between a tangent line, that is in contact with the ultrasound transducer array and in contact with the stepped surface at a position closest to the one side, and the ultrasound transducer array is defined as a first intersection, the first intersection is included within a range of ⅓ of the effective angle on a side of the proximal end.

15. The endoscope according to claim 14, further comprising:
an outlet port that is provided on the outer peripheral surface of the distal end part and that is open on the one side, and through which a treatment tool is led out;
a pipe line that is connected to the outlet port in the distal end part and through which the treatment tool is inserted; and
an observation window of an observation optical system that is provided on the stepped surface,
wherein, in a case where the distal end part is viewed from a side of the second direction, a second intersection, which is an intersection between a center line of a distal end part of the pipe line connected to the outlet port and the tangent line, is positioned closer to the proximal end side of the distal end part than the first intersection.

16. The endoscope according to claim 15,
wherein the second intersection is positioned between the ultrasound transducer array and the stepped surface.
